(19) United States
(12) Patent Application Publication
(10) Pub. No.: US 2013/0060288 A1
(43) Pub. Date: Mar. 7, 2013

(54) METHODS AND INSTRUMENTATION FOR ORTHOPEDIC SURGERY

(71) Applicants: W. Blake Rodgers, Jefferson City, MO (US); Eugene Shoshtaev, San Diego, CA (US); Lukas Eisermann, San Diego, CA (US); Joseph Riina, Fortville, IN (US); Randall Dryer, Austin, TX (US); Jim Youssef, Durango, CO (US); Mary Catherine Hardy, San Diego, CA (US)

(72) Inventors: W. Blake Rodgers, Jefferson City, MO (US); Eugene Shoshtaev, San Diego, CA (US); Lukas Eisermann, San Diego, CA (US); Joseph Riina, Fortville, IN (US); Randall Dryer, Austin, TX (US); Jim Youssef, Durango, CO (US); Mary Catherine Hardy, San Diego, CA (US)

(73) Assignee: NUVASIVE, INC., San Diego, CA (US)

(21) Appl. No.: 12/822,147

(22) Filed: Jun. 23, 2010

Related U.S. Application Data
(60) Provisional application No. 61/219,745, filed on Jun. 23, 2009.

(51) Int. Cl. A61B 17/88; A61B 17/56; A61B 17/80

(52) U.S. Cl. 606/281; 606/283; 606/86 B

(57) ABSTRACT

Surgical instruments and methods aimed at the proper placement and installation of textile-based orthopedic implants. More specifically, the surgical instruments include a flexible template, insertion instrument and multi-pronged awl. The flexible template may be formed of a combination of rigid and flexible materials and is deformable to match the contour of the target anatomy for subsequent placement of a textile-based orthopedic implant. The insertion instrument has a handle and a template end used for holding the flexible template and textile-based implant. The template end has one or apertures with hollow cylindrical extensions for holding the flexible template or textile-based implant and one or more cutout regions for better visualization and affixation of fixation screws. An awl is also provided with one or more prongs to initiate channels for the fixation screws. A method of implanting a textile-based orthopedic implant is also disclosed.

### CROSS-REFERENCES TO RELATED APPLICATIONS

The present application is a non-provisional application claiming the benefit of priority under 35 U.S.C. §119(e) to U.S. Provisional Application No. 61/219,745, filed Jun. 23, 2009, and entitled “ Methods and Instrumentation for Orthopedic Surgery,” the complete disclosure of which is hereby incorporated by reference in its entirety as if set forth fully herein.

### BACKGROUND OF THE INVENTION

I. Field of the Invention

The present invention relates to templates, instruments, and methods generally aimed at surgery and, more particularly, to templates, instruments, and methods aimed at the efficient and accurate installation of flexible implants.

II. Discussion of the Prior Art

Rigid plate implants have been used for many years to support bone fractures, reconstruct orthopedic structure across damaged ligaments, and to provide stability between fused spinal vertebrae. Rigid plate implants are usually affixed via fixation elements, such as bone screws, to nearby osseous tissues in order to restrict motion and to provide support during and after healing.

Although in many cases complete joint immobilization is preferred, in certain instances surgeons prefer to allow for retention of limited mobility across the affected joint during the course of post-operative fusion. Furthermore, in instances where the plate must conform to a bone, such as to protect a fracture or to support fused vertebrae, it can be challenging to manually shape the standard rigid surgical plate to custom fit the desired area.

Flexible textile-based orthopedic implants are therefore an alternative to rigid implants. The compliant nature of the textile-based implant provides the required flexibility to support a range of physiological movements, as opposed to a static fusion surgery. One example of a textile-based plate implant is described in commonly owned and co-pending U.S. patent application Ser. No. 12/274,345 entitled “Textile-Based Plate Implant and Related Methods”, filed Nov. 19, 2008, the entire contents of which are hereby incorporated by reference into this disclosure as if set forth fully herein. Textile-based orthopedic implants are suitable for use in many surgical applications, including but not limited to spinal fusion surgery.

When using sterile textile implants, it is important to first accurately determine which size or shape of implant to use for a given application before removing the implant from its sterile packaging. Trial size templates are often used during surgery to confirm which textile implant is an appropriate size and shape for the surgical application. Because the textile implants are flexible, a typical rigid trial size template mimicking the size and shape of the textile-based implant may not be appropriate to determine which implant is appropriate. A rigid trial size template cannot conform to the patient's anatomy as a flexible implant can. It may also be difficult to pass a rigid trial size template through the operative corridor to the target site, where a flexible textile implant would easily pass if folded or bent.

In addition to the flexible trial size template, an inserter instrument with various end sizes corresponding to the sizes of textile implants can be used to confirm which implant is an appropriate size and shape for the surgical application. The inserter instrument can also be used to guide an awl to make channels through the implant apertures for bone screws or other attachment means. The inserter instrument may be used to hold the flexible textile-based plate implant in place while the screws or other attachment means are installed.

### SUMMARY OF THE INVENTION

The templates and instruments describe herein may be constructed in any number of suitable fashions without departing from the scope of the present invention. The templates and instruments of the present invention are illustrated herein for use within the lumbar spine, but are suitable for use in other regions of the spine (e.g. cervical, thoracic), as well as for the repair of other bones and tissues containing fractures or needing reinforcement.

The templates are configured in various sizes analogous to the different sizes of textile-based orthopedic implants. The templates may be formed of a combination of rigid and flexible materials, or primarily of flexible materials. A variety of materials may be used to form the flexible portion of the templates, including but not limited to elastomer (e.g. silicone rubber), hydrogel, plastic mesh, plastic constructs, injectable fluids, and curable fluids. A variety of materials may be used to form the rigid portion of the templates, including but not limited to plastics and metals.

The template includes a first lateral end and a second lateral end, each having one or more apertures. Apertures on the trial size template correspond to screw apertures on the textile-based orthopedic implant where bone screws or other attachment means may be placed through to affix the implant to the osseous tissue. Lateral ends are composed of a rigid material. The template has a first coplanar side and a second coplanar side. A flexible section connects the lateral ends along an axis X extending between the coplanar sides. The flexible section enables the template to bend within the flexible section along the axis X.

According to another embodiment, the template includes a first lateral end and a second lateral end, each having one or more apertures. Apertures on the trial size template correspond to screw apertures on the textile-based orthopedic implant where bone screws or other attachment means may be placed through to affix the implant to the osseous tissue. Lateral ends are composed of a rigid material. The template has a first coplanar side and a second coplanar side. A flexible section connects the coplanar sides along an axis Y extending between the lateral ends. The flexible section enables the template to bend within the flexible section along the axis Y in either direction.

According to another embodiment, the template includes a first lateral end and a second lateral end, each having one or more apertures. Apertures on the trial size template correspond to screw apertures on the textile-based orthopedic implant where bone screws or other attachment means may be placed through to affix the implant to the osseous tissue. Lateral ends are composed of a rigid material. The template has a first coplanar side and a second coplanar side. A flexible section connects the coplanar sides along an axis Y extending between the lateral ends. The flexible section enables the template to bend within the flexible section along the axis Y in either direction. An additional flexible section also connects the lateral ends along an axis X extending between the coplanar sides. The flexible section enables the template to bend within the flexible section along the X axis in either direction.

According to another embodiment, the template includes lateral ends having apertures. Apertures on the trial size template correspond to apertures on the textile-based orthopedic implant where bone screws or other attachment means may be placed through to affix the implant to the osseous tissue. Lateral ends are composed of a rigid material. The template has a flexible hinge connecting the rigid lateral ends. The flexible hinge extends between a first coplanar side and a second coplanar side, along an axis X. The flexible hinge is made of a rigid material. The flexible hinge enables the template to bend at the flexible hinge along the axis X in either direction.

According to another embodiment, the template includes lateral ends having one or more apertures. Apertures on the trial size template correspond to apertures on the textile-based orthopedic implant where bone screws or other attachment means may be placed through to affix the implant to the osseous tissue. The template has a body connecting the lateral ends. The body and lateral ends are comprised entirely of or primarily of flexible material, enabling the template to bend linearly or torsionally in any direction as needed, similar to the flexibility of a textile-based orthopedic implant (not shown). Template may preferably be composed of a translucent or transparent polymer, which would increase intraoperative visibility.

In order to use the flexible trial size template, the template is introduced into a surgical site through use of any of a variety of suitable surgical instruments having the capability to engage the template, such as forceps. The template is capable of being used in minimally invasive surgical procedures, needing only a relatively small operative corridor for insertion when folded along the flexible section. After creating an operative corridor and preparing the surgical site using techniques commonly known and used in the art, the template is mated to an insertion device and advanced through the operative corridor toward the target surgical site. The template is positioned in a configuration suitable for the eventual installation of the textile-based orthopedic implant. If the selected template is not of an appropriate size for the desired target site, the template is removed from the surgical site and a different template is selected for trial sizing.

When the appropriate size of implant is determined, the template is removed from the surgical site and a corresponding textile-based orthopedic implant is selected and removed from its sterile packaging. The implant is then placed in the target site and affixed using techniques commonly known in the art.

The inserter instrument includes a handle, a template end, and an extension connecting the handle and template end. The inserter instrument is provided in a variety of template end sizes, analogous in size to the various sizes of textile-based orthopedic implants. The extension is of a length appropriate for insertion into a surgical operative corridor. The extension may be configured to be of an adjustable longitudinal length by telescoping or other means. The extension may include an elbow connector comprising an angle A to orient the template end in an ergonomic position relative to the handle. The elbow connector may be made of a flexible yet resistant material that allows the user to manipulate the elbow connector into a desired position, where it will remain until manipulated further.

The template end of the inserter instrument has a first lateral end and a second lateral end. Lateral ends include one or more screw apertures. At least one of lateral ends may have a cutout in place of a screw aperture. In one embodiment, both of lateral ends each have a cutout. One or more of the screw apertures may have one or more adjacent visualization apertures. Visualization apertures may follow a semi-circular path around the screw aperture, but other shapes permitting visualization of the underlying osseous tissues are contemplated.

The template end has a distal surface. The distal surface has hollow cylindrical extensions around the screw apertures, protruding in a distal direction. The cylindrical extensions are positioned to correspond to screw apertures on the textile-based orthopedic implant. The cylindrical extensions are configured to fit within the corresponding screw apertures of the textile-based orthopedic implant to hold the implant in place during insertion. The apertures allow the user to confirm where on the osseous tissues the bone screws or other attachment means are to be affixed.

In order to use the inserter instrument, a textile-based orthopedic implant is placed on the distal side of the appropriately sized template end of the inserter instrument. The screw apertures of the implant are placed around the cylindrical extensions. The inserter instrument and implant are then advanced into the surgical corridor to the target site. The inserter instrument is used to orient the implant in a desired configuration for affixation. The one or more screw apertures on the implant that is exposed by the cutout is affixed first in the manner described below, or other means commonly known in the art. The cutout is large enough to permit the screw to be externally driven by a tool, if needed. The inserter instrument holds the implant in place while screws are affixed through the apertures at the cutouts, preventing the implant from rotating with the screw as it is tightened. Next, the inserter instrument is removed from the implant and the remaining screws are placed through the implant.

The two-tipped awl has a handle for manipulating the awl. The handle is configured on its proximal end to withstand an impact by a hammer or other driving device. Distal to the handle is a connector extending between the handle and a bridge. The bridge is generally perpendicular to the handle and connector. At each lateral end of the bridge is an elbow connector. Each elbow connector is situated between the bridge and a prong. The prongs extending from each elbow connector are coplanar with the handle and connector. The prongs are positioned to correspond to the location of the screw apertures of the trial size templates, the screw apertures of the implant inserter instrument, and the screw apertures of textile-based orthopedic implants. Each prong terminates distally in a pointed tip. The width of the pointed tips is slightly less than or equal to the width of the bone screws or other means of attachment to be used. The pointed tips will enter the osseous tissue when the handle is hammered until the desired depth is reached, creating a channel for the placement of the bone screws.

The three-tipped awl has a handle for manipulating the awl. The handle is configured on its proximal end to withstand an impact by a hammer or other driving device. Distal to the handle is a connector extending between the handle and a bridge. The bridge is generally perpendicular to the handle and connector. At each lateral end of the bridge is an elbow connector. Each elbow connector is situated between the bridge and a prong. The three-tipped awl has an additional prong extending from the center of the bridge. The prongs are coplanar with the handle and connector. The prongs are positioned to correspond to the location of screw apertures of textile-based orthopedic implants. Each prong terminates distally in a pointed tip. The width of the pointed tips is slightly less than or equal to the width of the bone screws or other means of attachment to be used. The pointed tips will enter the osseous tissue when the handle is hammered until the desired depth is reached, creating a channel for the placement of the bone screws.

In order to use the awl, first the inserter instrument with a textile-based orthopedic implant attached, or the textile-based orthopedic implant, is placed on the surgical target in the desired position. Next, the awl is advanced through the surgical corridor to the target site. The pointed tips are positioned within the screw apertures of the implant. A hammer is used to impact the handle end that is protruding from the surgical corridor. The hammer is used until the pointed tips enter into the osseous tissue to the desired depth. The awl is then removed from the surgical corridor, and bone screws or other attachment means are affixed using the holes made by the pointed tips as path guides.

### BRIEF DESCRIPTION OF THE DRAWINGS

The invention will be more fully understood from the following detailed descriptions taken in conjunction with the accompanying drawings, in which:

FIG. 1 is a perspective view of an example of a flexible textile-based orthopedic implant.

FIG. 2 is a perspective view of an example of a flexible trial size template according to one embodiment of the present invention;

FIG. 3 is a perspective view of an example of a flexible trial size template according to another embodiment of the present invention;

FIG. 4 is a perspective view of an example of a flexible trial size template, according to another embodiment of the present invention;

FIG. 5 is a perspective view of an example of a flexible trial size template, according to another embodiment of the present invention;

FIG. 6 is a perspective view of an example of a flexible trial size template, according to another embodiment of the present invention;

FIG. 7 is a perspective view of an example of an inserter instrument, according to one embodiment of the present invention;

FIG. 8 is a perspective view of an example of a template end of an inserter instrument, according to another embodiment of the present invention;

FIG. 9 is a bottom view of the inserter instrument of FIG. 7;

FIG. 10 is a perspective view of an example of an awl instrument, according to one embodiment of the present invention; and

FIG. 11 is a perspective view of an example of an awl instrument, according to another embodiment of the present invention.

### DETAILED DESCRIPTION OF THE PREFERRED EMBODIMENT

Illustrative embodiments of the invention are described below for the purposes of understanding the principles of the invention. No limitation of the scope of the invention is therefore intended. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The invention disclosed herein boasts a variety of inventive features and components that warrant patent protection, both individually and in combination.

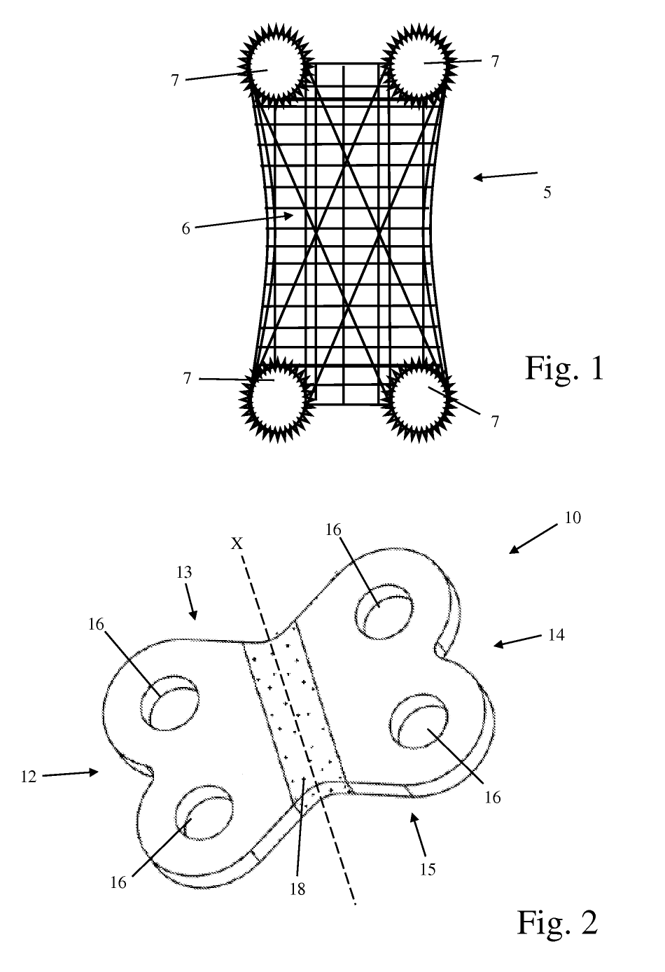

FIG. 1 illustrates an example of a flexible textile-based orthopedic implant 5 suitable for attachment to a pair of bone segments, for example a pair of vertebral bodies. One example of a textile-based plate implant is described in commonly owned and co-pending U.S. patent application Ser. No. 12/274,345 entitled “Textile-Based Plate Implant and Related Methods”, filed Nov. 19, 2008, the entire contents of which are hereby incorporated by reference into this disclosure as if set forth fully herein. The implant 5 includes a body 6 and a plurality of fixation apertures 7. Each fixation aperture 7 is configured to receive a fixation element (e.g. bone screw) to secure the implant 5 to the bony structures. The implant 5 shown by example includes four fixation apertures 7, however the specific number of fixation apertures 7 may vary according to the type of implant 5 used and the needs of the surgeon.

FIGS. 2-6 illustrate several examples of a flexible trial size template according to the present invention. The templates may be provided in various sizes analogous to the different sizes of textile-based orthopedic implants. The templates may be formed of a combination of rigid and flexible materials, or primarily of flexible materials. Any suitable material may be used to form the flexible portion of the templates, including but not limited to elastomer (e.g. silicone rubber), hydrogel, plastic mesh, plastic constructs, injectable fluids, curable fluids, and fibrous textile materials. The degree of flexibility provided is more than mere non-rigidity, such that the implant is capable of being substantially twisted or doubled over. Any suitable material may be used to form the rigid portion of the templates, including but not limited to plastics and metals.

FIG. 2 illustrates an example of a flexible trial size template 10 according to one embodiment of the present invention. The template 10 includes a first end portion 12 and a second end portion 14, each having one or more apertures 16. Apertures 16 on the trial size template 10 correspond to fixation apertures 7 on the textile-based orthopedic implant 5 of FIG. 1, where bone screws or other attachment means may be placed through to affix the implant to the osseous tissue. In all of the examples described herein, the number of apertures 16 is four, however it should be understood that the number of apertures 16 provided within the template 10 corresponds with the number of fixation apertures 7 of the textile-based implant 5, and therefore the template 10 may be provided with more or less than four apertures 16 without departing from the scope of the present invention. First and second end portions 12, 14 are composed of a rigid material, for example plastics and/or metal. The template 10 further has a first side 13 and a second side 15. A flexible section 18 is located between first and second end portions 12, 14 and connects the first and second end portions 12, 14 along an axis X extending between the sides 13, 15. The flexible section 18 enables the template to bend within the flexible section 18 along the axis X.

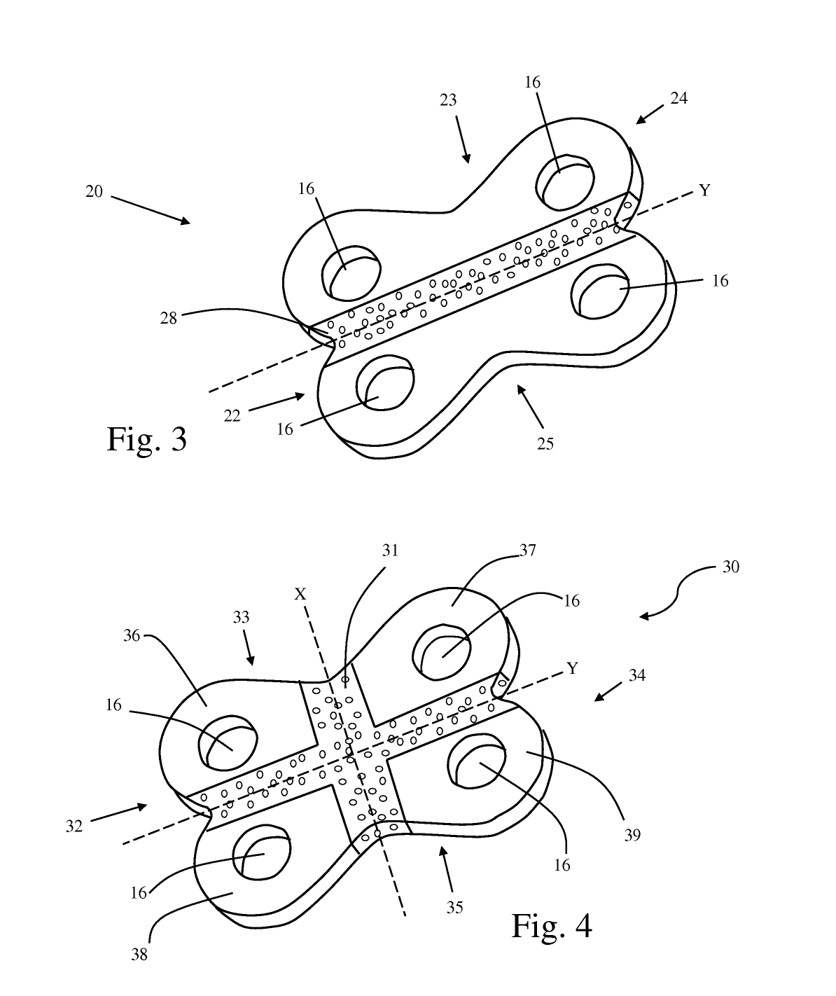

FIG. 3 illustrates an example of a flexible trial size template 20, according to another embodiment of the present invention. For simplicity of disclosure, elements of the various template examples described herein below that are substantially similar to elements of template 10 have been assigned the same callout numbers. The template 20 includes a first end portion 22 and a second end portion 24, each having one or more apertures 16. Apertures 16 on the trial size template 20 correspond to screw apertures 7 on the textile-based orthopedic implant 5 (FIG. 1). The template 20 has a first side portion 23 and a second side portion 25. A flexible section 28 connects the side portions 23, 25 along an axis Y extending longitudinally through the template 20. The flexible section 28 enables the template to bend within the flexible section 28 along the axis Y in either direction.

FIG. 4 illustrates an example of a flexible trial size template 30, according to yet another embodiment of the present invention. The template 30 is essentially divided into four zones of rigid material 36, 37, 38, 39 by the flexible section 31, which bisects the template 30 along two axes (X, Y). Each zone 36, 37, 38, 39 has one or more apertures 16. Apertures 16 on the trial size template 30 correspond to screw apertures 7 on the textile-based orthopedic implant (FIG. 1). The template 30 has a first side 33 and a second side 35 and a first lateral end 32 and a second lateral end 34. Flexible section 31 connects the sides 33, 35 along an axis Y extending between the lateral ends 32, 34. The flexible section 31 enables the template to bend within the flexible section 31 along the axis Y in either direction. Flexible section 31 also connects the lateral ends 32, 34 along an axis X extending between the sides 33, 35. The flexible section 31 enables the template to bend within the flexible section 31 along the X axis in either direction.

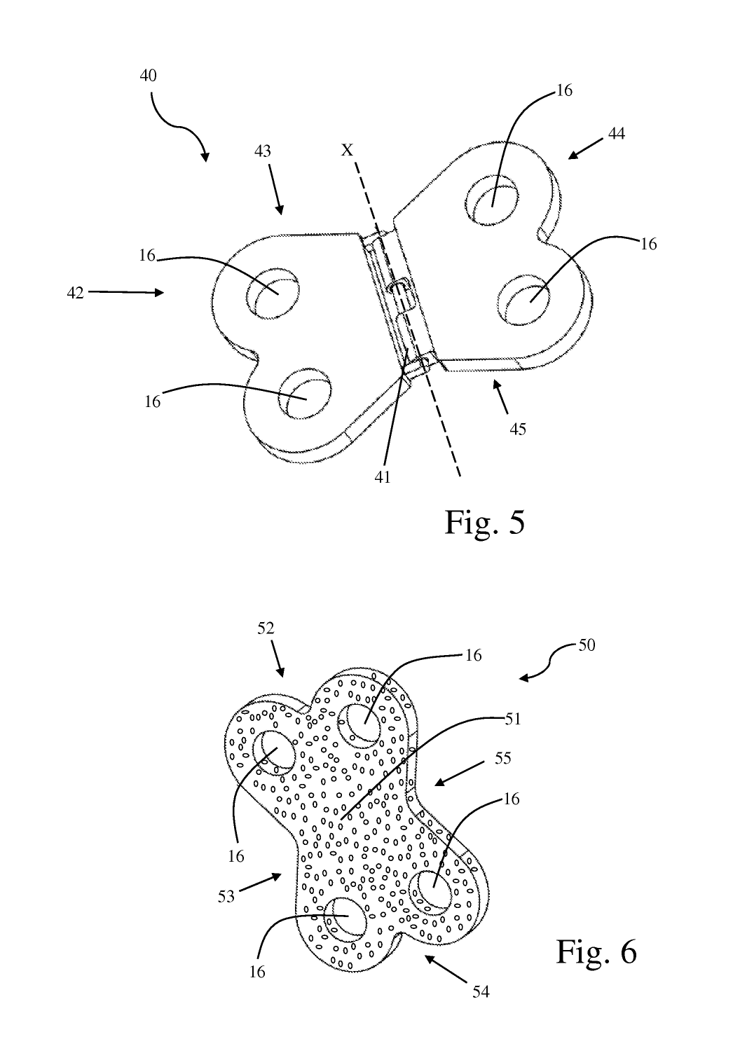

FIG. 5 illustrates an example of a flexible trial size template 40, according to another embodiment of the present invention. The template 40 includes lateral ends 42, 44 having apertures 16. Apertures 16 on the trial size template 40 correspond to apertures on the textile-based orthopedic implant 5 (FIG. 1) where bone screws or other attachment means may be placed through to affix the implant to the osseous tissue. Lateral ends 42, 44 are composed of a rigid material. The template 40 has a hinge 41 connecting the rigid lateral ends 42, 44. The hinge 41 extends between a first side 43 and a second side 45, along an axis X. The hinge 41 is made of a rigid material. The hinge 41 enables the template to bend at the hinge 41 along the axis X in either direction.

FIG. 6 illustrates a flexible trial size template 50, according to one embodiment of the present invention. The template 50 includes lateral ends 52, 54 having one or more apertures 16. Apertures 16 on the trial size template 50 correspond to apertures on the textile-based orthopedic implant 5 (FIG. 1) where bone screws or other attachment means may be placed through to affix the implant to the osseous tissue. The template 50 has a body 51 extending between the lateral ends 52, 54 and the sides 53, 55. The body 51 and lateral ends 52, 54 are comprised entirely of or primarily of flexible material, enabling the template 50 to bend linearly or torsionally in any direction as needed, similar to the flexibility of the textile-based orthopedic implant 5. Template 50 may preferably be composed of a translucent or transparent polymer, which would increase intraoperative visibility.

In order to use the flexible trial size template 10, 20, 30, 40, and 50, shown in FIGS. 2-6, the template is introduced into a surgical site through use of any of a variety of suitable surgical instruments having the capability to engage the template, such as forceps. The template is capable of being used in minimally invasive surgical procedures, needing only a relatively small operative corridor for insertion when folded along the flexible section. After creating an operative corridor and preparing the surgical site using techniques commonly known and used in the art, the template is mated to an insertion device and advanced through the operative corridor toward the target surgical site. The template is positioned in a configuration suitable for the eventual installation of the textile-based orthopedic implant. If the selected template is not of an appropriate size for the desired target site, the template is removed from the surgical site and a different template is selected for trial sizing.

When the appropriate size of implant is determined, the template is removed from the surgical site and a corresponding textile-based orthopedic implant is selected and removed from its sterile packaging. The implant is then placed in the target site and affixed using techniques commonly known in the art.

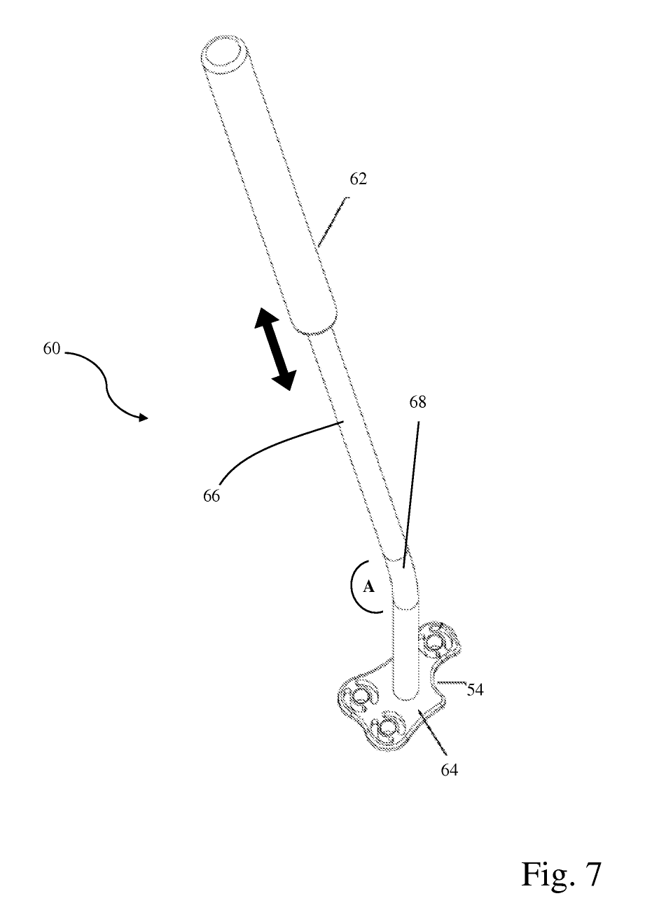

FIGS. 7-9 illustrate an example of an inserter instrument 60 according to one embodiment of the present invention. The inserter instrument 60 includes a handle 62, a template end 64, and an extension 66 connecting the handle 62 and template end 64, as illustrated in FIG. 7. The inserter instrument 60 may be provided in a variety of template end 64 sizes, analogous in size to the various sizes of textile-based orthopedic implants 5 (FIG. 1). The extension 66 is of a length appropriate for insertion into a surgical operative corridor. The extension 66 may be configured to be of an adjustable longitudinal length by telescoping or other means, as illustrated by the arrows in FIG. 7. The extension may include an elbow connector 68 comprising an angle A to orient the template end 64 in an ergonomic position relative to the handle 62. The elbow connector 68 may be made of a flexible yet resistant material that allows the user to manipulate the elbow connector 68 into a desired position, where it will remain until manipulated further.

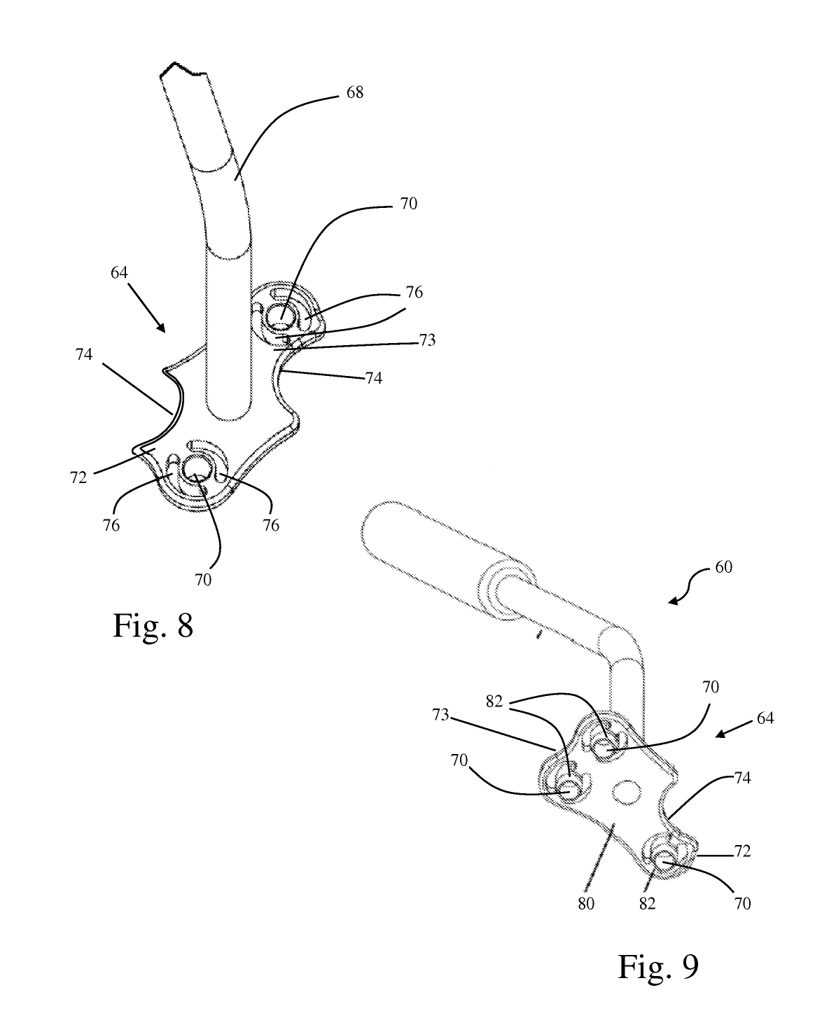

FIGS. 8-9 further illustrate the template end 64 of the inserter instrument 60. The template end 64 has a first lateral end 72 and a second lateral end 73. Lateral ends 72, 73 include one or more screw apertures 70. At least one of lateral ends 72, 73 may have a cutout 74 in place of a screw aperture 70. In one embodiment, illustrated in FIG. 8, both lateral ends 72, 73 have a cutout 74. The cutouts 74 serve multiple purposes. First, it allows for an inserter with a smaller overall profile such that it can fit through smaller operative corridors. Second, it reduces the material required so that costs may be reduced. Another benefit of the cutout 74 is that it increases visibility of the surgical target site by the surgeon during the procedure. One or more of the screw apertures 70 may have one or more adjacent visualization apertures 76. Visualization apertures 76 may follow a semi-circular path around the screw aperture 70, as illustrated in FIGS. 7-9, but other shapes permitting visualization of the underlying osseous tissues are contemplated.

The template end 64 has a distal surface 80, as shown in FIG. 9. The distal surface side 80 has hollow cylindrical extensions 82 around the screw apertures 70, protruding in a distal direction. The cylindrical extensions 82 are positioned to correspond to screw apertures 7 on the textile-based orthopedic implant 5 (FIG. 1). The cylindrical extensions 82 are configured to fit within the corresponding screw apertures of the textile-based orthopedic implant to hold the implant in place during insertion. The apertures 70 allow the user to confirm where on the osseous tissues the bone screws or other attachment means are to be affixed.

In order to use the inserter instrument 60, a textile-based orthopedic implant is placed on the distal side of the appropriately sized template end 64 of the inserter instrument 60. The screw apertures 7 of the implant 5 are placed around the cylindrical extensions 82. The inserter instrument 60 and implant 5 are then advanced into the surgical corridor to the target site. The inserter instrument 60 is used to orient the implant 5 in a desired configuration for affixation. The one or more screw apertures 7 on the implant that is exposed by the cutout 74 is affixed first in the manner described below, or other means commonly known in the art. The cutout 74 is large enough to permit the screw to be externally driven by a tool, if needed. The inserter instrument 60 holds the implant in place while screws are affixed through the apertures 7 at the cutouts 74, preventing the implant from rotating with the screw as it is tightened. Next, the inserter instrument 60 is removed from the implant and the remaining screws are placed through the implant.

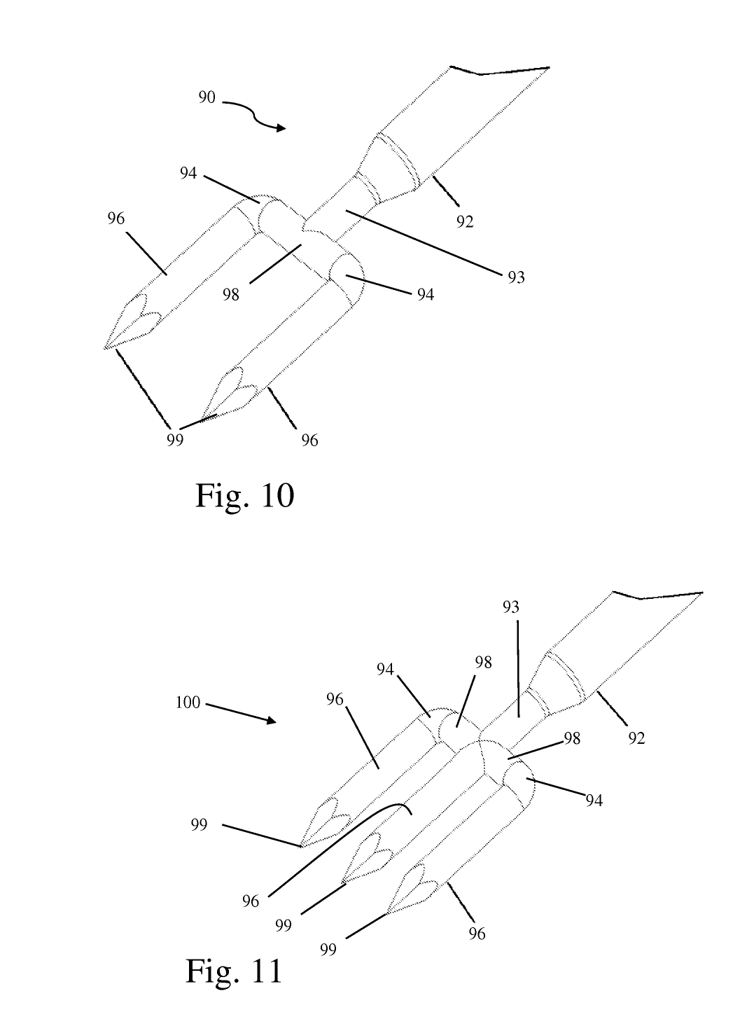

FIG. 10 illustrates an example of a two-tipped awl 90 of the present invention. The awl 90 has a handle 92 for manipulating the awl 90. The handle 92 is configured on its proximal end (not shown) to withstand an impact by a hammer (for example a mallet or slap hammer) or other driving device. Distal to the handle 92 is a connector 93 extending between the handle 92 and a bridge 98. The bridge 98 is generally perpendicular to the handle 92 and connector 93. At each lateral end of the bridge 98 is an elbow connector 94. Each elbow connector 94 is situated between the bridge 98 and a prong 96. The prongs 96 extending from each elbow connector 94 are coplanar with the handle 92 and connector 93. The prongs 96 are positioned to correspond to the location of the screw apertures 16 of the trial size templates 10, 20, 30, 40, and 50, the screw apertures 70 of the implant inserter instrument 60, and the screw apertures 7 of textile-based orthopedic implant 5. Each prong 96 terminates distally in a pointed tip 99. The width of the pointed tips 99 is slightly less than or equal to the width of the bone screws or other means of attachment to be used. The pointed tips 99 will enter the osseous tissue when the handle 92 is hammered until the desired depth is reached, creating a channel for the placement of the bone screws.

FIG. 11 illustrates an example of a three-tipped awl 100 of the present invention. For simplicity of disclosure, elements of awl 100 that are substantially similar to elements of awl 90 have been assigned the same callout numbers. The awl 100 has a handle 92 for manipulating the awl 100. The handle 92 is configured on its proximal end (not shown) to withstand an impact by a hammer or other driving device. Distal to the handle 92 is a connector 93 extending between the handle and a bridge 98. The bridge 98 is generally perpendicular to the handle 92 and connector 93. At each lateral end of the bridge 98 is an elbow connector 94. Each elbow connector 94 is situated between the bridge 98 and a prong 96. The three-tipped awl 100 has an additional prong 96 extending from the center of the bridge 98. The prongs 96 are coplanar with the handle 92 and connector 93. The prongs 96 are positioned to correspond to the location of screw apertures 7 of textile-based orthopedic implant 5 (FIG. 1). Each prong 96 terminates distally in a pointed tip 99. The width of the pointed tips 99 is slightly less than or equal to the width of the bone screws or other means of attachment to be used. The pointed tips 99 will enter the osseous tissue when the handle 92 is hammered until the desired depth is reached, creating a channel for the placement of the bone screws.

In order to use the awls 90, 100, first the inserter instrument 60 with a textile-based orthopedic implant 5 attached, or the textile-based orthopedic implant 5, is placed on the surgical target in the desired position. Next, the awl is advanced through the surgical corridor to the target site. The pointed tips 99 are positioned within the screw apertures of the implant. A hammer is used to impact the handle end that is protruding from the surgical corridor. The hammer is used until the pointed tips 99 enter into the osseous tissue to the desired depth. The awl 90, 100 is then removed from the surgical corridor, and bone screws or other attachment means are affixed using the holes made by the pointed tips 99 as path guides.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the invention is to cover all modifications, equivalents, and alternatives falling within the spirit and scope of the invention as defined herein.

1. A flexible trial size template, comprising:
a top, a bottom, and two opposing sides;
a first zone composed of a rigid material and containing one or more apertures, a second zone composed of a rigid material and containing one or more apertures, a third zone composed of a rigid material and containing one or more apertures, and a fourth zone composed of a rigid material and containing one or more apertures, and
a flexible body intersecting the trial size template along two intersecting axes, the first axis extending from the top to the bottom of the template, the second axis extending between first and second opposing sides, each of the two intersecting axes connectedly dividing two zones of the flexible trial size template from two other zones of the flexible trial size template, the flexible body being composed of a flexible material to enable the trial size template to flexibly adapt to a surgical site.

2. The flexible trial size template of claim 1, wherein the rigid material comprising at least one of plastic, metal, or ceramic.

3. The flexible trial size template of claim 1, wherein the flexible material comprises at least one of textile, polymer, elastomer, silicone rubber, plastic mesh, plastic construct, hydrogel, injectable fluid, or curable fluid.

4. The flexible trial size template of claim 3 wherein the flexible material has transparent properties to enable increased intraoperative visibility.

5.-12. (canceled)

13. A method of implanting a textile-based implant comprising the steps of:
selecting a flexible trial size template for determination of an appropriately shaped and sized textile-based implant for implantation;
inserting the flexible trial size template coupled to an insertion device through a minimally invasive operative corridor to a target surgical site;
conforming the flexible trial size template to a target anatomy to approximate subsequent placement of a textile-based orthopedic implant;
removing the flexible trial size template coupled to the insertion device from the surgical site and from the insertion device;
selecting an appropriately sized textile-based orthopedic implant based on the flexible trial size template;
removably attaching the textile-based implant to the inserter instrument by attachably engaging one more apertures located on the textile-based implant with one or more hollow cylindrical extensions distally protruding from a template end of the inserter instrument, the template end of the inserter instrument having one or more cutouts replacing one or more apertures located on the template end of the inserter instrument and allowing direct access to one or more exposed apertures located on the textile-based implant;
advancing the textile-based implant removably attached to the inserter instrument through the minimally invasive operative corridor towards the target surgical site;
orienting the inserter instrument to place the textile-based implant in a desired configuration for affixation;
advancing one or more fixation screws through the one or more cutout regions of the template end of the inserter instrument and through one or more apertures of the textile-based implant;
affixing the one or more fixation screws into osseous tissue through the one or more apertures of the textile-based implant via the one or more cutout regions, to prevent the textile-based implant form rotating with the one or more fixation screws during tightening of the one or more fixation screws; and
removing the inserter instrument from the orthopedic implant and the surgical site to allow for placement of one or more fixation screws directly through the textile-based implant.

14. The method of implanting a textile-based implant of claim 13, further comprising the step of using an awl having one or more prongs to initiate channels for the one or more fixation screws prior to affixation of the one or more fixation screws.
